(19) United States
(12) Patent Application Publication
(10) Pub. No.: US 2016/0230914 A1
(43) Pub. Date: Aug. 11, 2016

(54) Slip Connector with Alarm

(71) Applicant: Sanjay K Roy, Palmetto Bay, FL (US)

(72) Inventor: Sanjay K Roy, Palmetto Bay, FL (US)

(21) Appl. No.: 15/019,925

(22) Filed: Feb. 9, 2016

Related U.S. Application Data
(60) Provisional application No. 62/114,711, filed on Feb. 11, 2015.

(51) Int. Cl. F16L 37/252

(52) U.S. Cl. CPC F16L 37/252

(57) ABSTRACT

A luer slip connector assembly where the connector must disengage if the axial force on two connected tubes rises above a predetermined value, while simultaneously activating an automatic alarm indicating the disengagement, is disclosed. It can also be used in other applications incorporating “slip” connectors, for example, couplings in fluids, electronic/electric, optical and other systems.

### RELATED APPLICATIONS

This application claims the benefit of U.S. Provisional Patent Appl. Ser. No. 62/114,711 filed on Feb. 11, 2015—the contents of which are incorporated by reference herein.

### FIELD OF THE INVENTION

The present invention relates to luer connectors that are widely used in biomedical applications for coupling two tubings employed for carrying various types of fluids. It is specifically designed for uses where the connector must disengage if the axial force on the two connected tubes rises above a predetermined value, while simultaneously activating an automatic alarm indicating the disengagement. It can also be used in other applications incorporating “slip” connectors, for example, couplings in fluids, electronic/electric, optical and other systems.

### BACKGROUND OF THE INVENTION

Luer lock connectors are used in many biomedical systems to couple two fluid carrying tubes. A major advantage of these connectors is their lockable coupling-decoupling feature that can be easily manipulated by a single user. The connectors themselves comprise standardized mating conical coupling surfaces (e.g. ISO 594, 80369) designed to prevent leaks under normal operating pressures, which are relatively low (typically below 300 kPa). The locking feature comprises a double start right handed female threaded nut on one connector that locks on to a corresponding male thread or lug feature on the other connector. If the locking function is not required, the locking features are eliminated and the resulting “luer slip” connectors are held together by friction at the conical interfaces of the connectors themselves.

The locking feature is usually necessary for normal use. However, in many applications, it is useful if the connectors disengage when the axial forces on the tubes being connected become too high. An example of this is associated with the use of catheters that collect urine or other bodily fluids over extended periods of time. In this type of application, the catheter is attached to the human body with the fluid flowing out through a discharge tube into a collection bag for ultimate disposal. Since the collection bag may need to be replaced well before the catheter itself, the typical arrangement comprises a catheter-tubing assembly and a collection bag-tubing assembly that are coupled together using luer lock connectors. Though this approach is functional, it often poses a major problem: if the tubing gets caught or tangled up accidentally in clothing, limbs, bedding, or other similar object, a significant axial/pulling force can occur on the overall assembly. This may result in the catheter being pulled out of the body causing injury and harm to the patient.

A simple solution here is to use luer slip connectors instead of lock connectors. However, there is a problem with this approach, viz. the fluid begins to leak when the connectors disengage due to excessive forces. When this occurs, the user/patient is often not aware of this for an extended period of time, and extensive cleanup may be required when disengagement is first noted. An obvious approach here is to use an alarm system that can notify the user (or care-giver) of the disengagement. However, low-cost, compact, disposable alarms that can be used for such a configuration are not available. As a result, luer lock connectors continue to be used even when their disadvantages cause problems during use.

Given the above, it is clear that there is a need for a connector that can disengage when the axial pulling forces become excessive (i.e. rises above a predetermined/maximum value) and that can provide an alarm when this disengagement occurs. It is also very important that the overall connector-alarm assembly be simple, compact and low cost so that it can be disposable, thereby permitting its use in biomedical applications. Note that similar low cost connector-alarm assemblies can also be used in other fluid, electrical and optical systems where it is often inefficient and expensive to incorporate localized alarms in large networks.

### Description of Prior Art

Luer connectors carrying various types of fluids have been proposed, e.g. U.S. Pat. Nos. 4,294,250, 5,312,377. International and national standards for such connectors exist for both locking and slip type luer connectors for use in different applications. However, low cost, slip connectors with integrated alarms have yet to be described in prior art.

### SUMMARY OF THE INVENTION

The object of this invention is to provide a connector assembly that overcomes the disadvantages of current luer connectors as mentioned above. The invention achieves this by combining the following:

a luer slip connector assembly that provides the basic connection between the two fluid lines,

a battery powered, low cost alarm assembly, and

an integrated triggering mechanism that activates the alarm when the luer assembly separates/disengages during use.

This invention incorporates the following novel features:

1. The alarm has specific design characteristics that make it compact and low cost.

2. The alarm is integrated with the connectors such that it automatically turns on when the connector assembly disconnects.

3. The integrated assembly is designed such that the connector detaches when the axial pulling forces are greater than a predetermined maximum value or range.

For catheter type applications described above, it is expected that the detachment force requirement will be of the order of 300-400 grams or lower. For other applications, it may be different.

### BRIEF DESCRIPTION OF THE DRAWINGS

FIG. 1 is a schematic section view of a preferred embodiment of the present invention when the male and female connector subassembly are in mated position. FIG. 2 is a schematic section view without the male connector inserted in the female connector subassembly.

### DETAILED DESCRIPTION

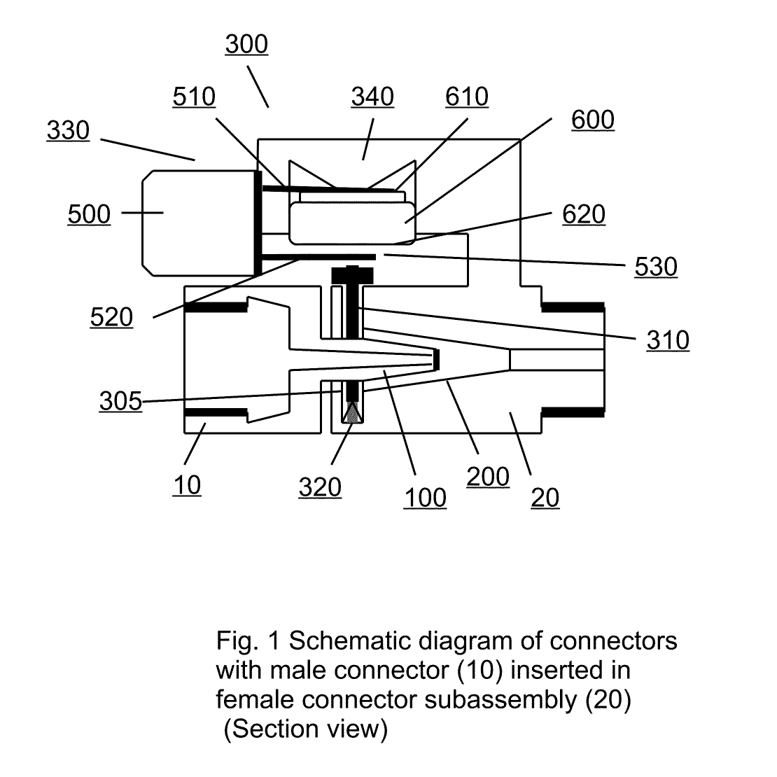

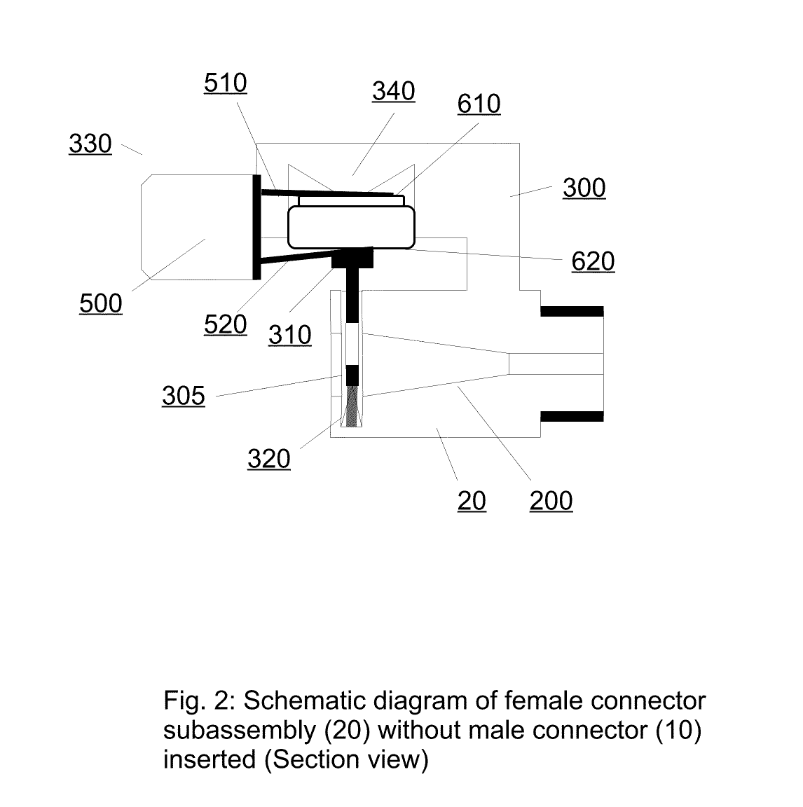

FIG. 1 is a schematic view of a preferred embodiment of the present invention. It consists of the two parts/subassemblies, a male side connector (10) and a female side connector subassembly (20), that together form the overall assembly.

The male side connector (10) comprises a standard slip luer (male) connector (100) that connects with the female side connector assembly (20). The female side assembly comprises a standard slip luer (female) connector (200) that is integrated with the switching and alarm assembly (300). The switching and alarm assembly (300) comprises of a switching plate (310), a spring (320), an alarm (330) and a stop feature (340). The switching plate (310) is activated by the compression spring (320), and is positioned such that it can move relative to the female luer connector (200) in a slot/guide/mating feature (305) of the assembly. When the male side connector (100) is coupled to the female side connector (200) as shown in FIG. 1, the switching plate (310) is prevented from moving in its guide (305) by the male connector (100) that keeps it in its lower position.

The alarm (330) itself comprises only two components, an electromagnetic buzzer/indicator module (500) and a zinc-air battery (600). The through-hole mount electromagnetic buzzer (500) is the primary component in the preferred embodiment. It comprises a single module that includes both an electromagnetic audio transducer and its drive circuitry. This reduces the overall size and cost by eliminating the circuitry required to drive a separate electromagnetic transducer. The module (500) is also selected such that its input voltage matches the output of the second component, a zinc-air cell/battery (e.g. 1.2-1.5 V for common zinc-air cells).

The zinc-air cell/battery (600) is selected to provide power to drive the buzzer module. Unlike current alarm systems that use other standard batteries (i.e. alkaline or lithium ion), a zinc-air battery is specifically used in this invention for a number of important reasons:
its cost is lower than that of other batteries of the same capacity
it is smaller and lighter other batteries of the same capacity

The alarm (330) is integrated (or mounted in) the stop feature such that one of the (i.e. either the positive or the negative) leads (510) of the buzzer module (500) in direct electrical contact with corresponding terminal (610) of the zinc-air battery, and the other lead (520) is positioned adjacent to the second terminal (620) with a gap (530) between them so that there is no electrical contact.

In its connected configuration, the male connector (100) maintains the switching plate in the lower position, thereby compressing the spring (320). In this state, the alarm (330) is turned off since its electrical circuit is open due to the gap (530) between the second lead (520) and the battery terminal (620). When the male connector is pulled out (see FIG. 2), the spring (320) pushes the switching plate (310) towards the stop feature until until it makes contact with the alarm/stop (330/340). At this point, the second lead (520) comes in direct contact with the battery terminal (620) and the alarm (330) turns on. To turn off the alarm (330), the user must now push the switching plate (310) down and re-insert the male connector (100) back into the female connector (200).

It is important to note the unique features of the invention:

a. The cost and size will be very low. Only two electrical components are used and all contacts can be done mechanically by enforcing using suitable tolerances in the male side connector and a female side connector assembly design. Note that electrical switching is also achieved by using the lead of a component itself. Thus, individual components such as PCBs, electromechanical switches, battery holders and other electronic parts not used, and cost adders related to PCB assembly and packaging are eliminated.

b. The disconnect force in this configuration is determined by two factors: friction between the luer connectors and the additional frictional force between the switching plate and the male connector. Depending on the disconnect force requirement, the spring force (and thereby, the friction force) can be changed for connectors to be used in different applications. Alternatively, the surface finish (or material) of contacting portion of the switching plate and/or the male connector can be modified to adjust the friction force as required.

The overall connector assembly described above can be attached to the corresponding tubing or hoses, using standard fittings (e.g. hose, tube, pipe, etc.). Thus, different versions of the assembly can be made with different threads, fittings, etc., at the open ends to accommodate corresponding fittings for different applications. For biomedical applications as proposed, luer lock fittings may be incorporated on the two open ends so that the overall connector assembly can used together with existing luer lock connectors.

The present invention is not limited to the above described embodiments, and different variations and modifications that may be evident to one skilled in the art are possible without departing from its overall scope. For example, specific features that will enhance manufacturability of the connector components can and will be incorporated into the above embodiments to ensure that it can be manufactured at a reasonable cost (e.g drafts on other surfaces, rounds, etc.). Other variations of the above configuration include the following:

a. The second lead can be mounted directly on the surface of the switching plate. This will ensure that it cannot come in contact with the second battery contact inadvertently during use when the connectors are still engaged. In this approach, the switching plate can be made of an electrically conductive material to simplify the design/manufacturability.

b. The alarm can be integrated with the switching plate instead of the stop feature. Corresponding to (a) above, the second lead can now be mounted on the surface of the stop feature if necessary (which can also be made of a conductive material).

c. Battery contacts or other other extensions may be attached to the leads of the buzzer (e.g. via crimping, spot welding, soldering, etc.) to improve the electrical contact between the buzzer and the battery if necessary. This may be appropriate if the length of the buzzer leads are too small. Alternatively, contact pins or receptacles (possibly with battery holders) may be integrated with the battery itself, if the resulting higher costs are acceptable.

d. The switching plate and guide design can be different from the one shown in the figure: it is not necessary for the switching plate to (partially) close the access of the male connector to the female connector when the connectors are disconnected. Alternative designs may allow coupling in both cases—the critical issue is the switch locking function provided by the male connector when it is coupled to the female connector.

e. The entire switching and alarm assembly can be positioned horizontally (i.e. parallel to the direction of insertion of the connectors) instead of vertically. In this case, the spring will be placed parallel to the connectors (as opposed to perpendicular in the configuration that is described above in the preferred embodiment). Thus, the spring force can be used to oppose the connection force, and the disconnect force can be less than the friction between the slip connectors. Note that for this type of configuration, the switching and alarm assembly can also be integrated with the male connector instead of the female connector as in the embodiment described above.

f. A tension spring arrangement can be used as the force applicator instead of a compression spring. In this case, the spring would be in its stretched position when the alarm is off, and would contract while initiating contact. More than one spring can also be used to obtain the required force or force distribution. Alternatively, permanent magnets can be used to provide the actuating force instead of a spring. Note also that the spring(s) can be integrated with the switching plate (i.e. they comprise a single part), particularly if the switching plate is made of a metallic material: this may help reduce costs further.

g. Instead of an electromagnetic buzzer module, other transducers or alarm modules may be used in this invention. Some alternatives include piezoelectric buzzers, LED modules (or other light emitting devices), vibratory motors or even low power RF transmitters, etc. Note that the power source (i.e. the battery) may need to be changed so as to match the transducer requirement. The overall construction however will be similar to the one described above.

h. As mentioned previously, the invention can be used with other connectors as well as applications such as security containers (where an alarm is often necessary to indicate that they are opened). The only requirement here is the presence of two moving parts that have a mated (connected) and an un-mated (disconnected) position during use. Note also that the invention can be designed to operate in the reverse mode (i.e. the alarm ON when mated and OFF when disconnected) with minor obvious modifications.

i. In an advanced embodiment, the luer connectors can incorporate secondary connecting features with valves that are opened-closed by coupling-decoupling the two components. This will provide a system with low- or no-drip functionality that may be required in some applications.

j. More complex versions may use PCB based designs with audio (or other) transducers and specially designed drive circuits to allow for more flexibility in use and application. Rechargeable batteries with charging circuits, electromechanical switches, etc. are additional options that can be considered, though these can be expected to increase cost and size.

What is claimed is:

1. A luer connector assembly comprising a male luer slip connector and a female slip connector subassembly,
said female slip connector subassembly comprising a female luer slip connector and a switching and alarm assembly,
said male luer slip connector and male slip luer connector having an axis of connection when connected together,
said switching and alarm assembly comprising a guide feature, a switching plate, a spring, an alarm and a stop feature.
said guide feature comprising a guideway, a slot and an opening permitting said male luer connectors to access said female luer connector,
said guideway and slot oriented perpendicular to the said axis of connection of said luer connectors,
said slot having at least one opening allowing said switching plate to be inserted within and permitting said switching plate to move within said slot and guide feature,
said switching plate comprising an opening permitting said male luer connectors to access said female luer connector,
said male luer connector preventing said switching plate from moving in said guide towards said stop feature when said male connector is inserted in said female connector,
said spring positioned such that it can force said switching plate to move in said guide feature when said male luer connector is not inserted in said female luer connector,
said alarm comprising an alarm module and a battery,
said alarm module comprising an electromagnetic buzzer with two external connector leads, one positive and one negative,
said battery providing electric voltage and power to drive said alarm module,
said alarm module and battery positioned such that
one of positive or negative lead of said alarm module is in electrical connection with corresponding terminal of said battery and
the other of said positive or negative lead of said alarm module is adjacent to other corresponding terminal of said battery but not in electrical connection with it when said male luer connector is connected to said female luer connector,
said alarm mounted on one of said switching plate or said stop feature,
with said spring forcing said switching plate towards said stop feature when said male luer connector is disconnected from said female luer connector, thereby moving said other lead of said alarm module towards said battery such that it makes electrical connection with said other corresponding terminal of said battery.

2. The luer connector assembly of claim 1, wherein a battery contact is attached to at least one of said leads of said alarm module and said battery contact is used to make electrical contact with corresponding contact of said battery.

3. The luer connector assembly of claim 1, wherein said other of said positive or negative lead of said alarm module is attached to to said switching plate.

4. The luer connector assembly of claim 3, wherein said switching plate is made of an electrically conductive material.

5. The luer assembly of claim 1, wherein said spring is integrated with said switching plate.

6. The luer assembly of claim 1, wherein at least one of the contact surfaces between the switching plate and male luer connector is modified to enhance or control the sliding friction force between them.

7. A slip connector assembly comprising a male slip connector, a female slip connector and a switching and alarm subassembly,
said switching and alarm subassembly being integrated with one of said male slip connector or female slip connector,
said switching and alarm subassembly comprising a switching plate, a guide feature, a force applicator, an alarm and a stop feature.
said switching plate positioned such that it can move relative to said female luer connector in said guide feature,
said force applicator positioned such that it can move said switching plate in its guide feature,
said alarm comprising an alarm module and a battery,
said alarm module comprising a transducer with any required drive circuitry, and two external connector leads,
said battery providing electric voltage and power to drive said alarm module,
said alarm module and battery positioned such that
one of positive or negative lead of said alarm module is in electrical connection with corresponding terminal of said battery and
the other of said positive or negative lead of said alarm module is adjacent to other corresponding terminal of said battery but not in electrical connection with it when said male connector is connected to said female connector,
said alarm mounted on one of said switching plate or said stop feature,
said switching plate and alarm subassembly comprising features that prevent said switching plate from moving in said guide towards said stop feature when said male connector is inserted in said female connector,
with said force applicator forcing said switching plate towards said stop feature when said male connector is disconnected from said female connector such that said other lead of said alarm module makes electrical connection with said other corresponding terminal of said battery.

8. The slip connector assembly of claim 1, wherein said alarm module is an electromagnetic buzzer and said battery is a zinc-air battery.

9. The slip connector assembly if claim 1, wherein said force applicator is one of a compression spring, a tension spring or a permanent magnet.

10. The slip connector assembly of claim 1, wherein said alarm module is one of a piezoelectric buzzer, a electromagnetic buzzer, a LED module or an RF transmitter module.

11. The slip connector assembly of claim 1, wherein battery contacts are attached to said leads of said alarm module such that said battery contacts are used to make electrical contact with corresponding contacts of said battery.

12. The slip connector assembly of claim 1, wherein said slip connectors may be one of fluids, electrical or optical type.

13. A mating assembly with alarm comprising two mating components and a switching and alarm subassembly,
said switching and alarm subassembly being integrated with one of said mating components,
said switching and alarm subassembly comprising a switching plate, a guide feature, a force applicator, an alarm and a stop feature.
said switching plate positioned such that it can move relative to said female luer connector in said guide feature,
said force applicator positioned such that it can move said switching plate in its guide feature,
said alarm comprising an alarm module and a battery,
said alarm module comprising a transducer with any required drive circuitry, and two external connector leads,
said battery providing electric voltage and power to drive said alarm module,
said alarm module and battery positioned such that
one of positive or negative lead of said alarm module is in electrical connection with corresponding terminal of said battery and
the other of said positive or negative lead of said alarm module is adjacent to other corresponding terminal of said battery but not in electrical connection with it when said two mating components are connected,
said alarm mounted on one of said switching plate or said stop feature,
said switching plate and alarm subassembly comprising features that prevent said switching plate from moving in said guide towards said stop feature when said two mating components are connected,
with said force applicator forcing said switching plate towards said stop feature when said two mating components are disconnected from each other such that said other lead of said alarm module makes electrical connection with said other corresponding terminal of said battery

14. The mating assembly with alarm of claim 1, wherein said alarm module is an electromagnetic buzzer and said battery is a zinc-air battery.

15. The mating assembly with alarm of claim 1, wherein said force applicator is one of a compression spring, a tension spring or a permanent magnet.

16. The mating assembly with alarm of claim 1, wherein said alarm module is one of a piezoelectric buzzer, a electromagnetic buzzer, a LED module or an RF transmitter module.

17. The mating assembly with alarm of claim 1, wherein battery contacts are attached to said leads of said alarm module such that said battery contacts are used to make electrical contact with corresponding contacts of said battery.

18. The mating assembly with alarm of claim 1, wherein said mating assembly comprises one of a mating slip connector assembly or mating mechanical container parts.

19. The slip connector assembly of claim 6, wherein said slip connectors may be one of fluids, electrical or optical type.

20. An apparatus with a mating assembly comprising two mating components and a switching and alarm subassembly,
said switching and alarm subassembly being integrated with one of said mating components,
said switching and alarm subassembly comprising a switching plate, a guide feature, a force applicator,
an alarm and a stop feature.
said switching plate positioned such that it can move relative to said female luer connector in said guide feature,
said force applicator positioned such that it can move said switching plate in its guide feature,
said alarm comprising an alarm module and a battery,
said alarm module comprising a transducer with any required drive circuitry, and two external connector leads,
said battery providing electric voltage and power to drive said alarm module,
said alarm module and battery positioned such that
one of positive or negative lead of said alarm module is in electrical connection with corresponding terminal of said battery and
the other of said positive or negative lead of said alarm module is adjacent to other corresponding terminal of said battery but not in electrical connection with it when said two mating components are connected,
said alarm mounted on one of said switching plate or said stop feature,
said switching plate and alarm subassembly comprising features that prevent said switching plate from moving in said guide towards said stop feature when said two mating components are connected,
with said force applicator forcing said switching plate towards said stop feature when said two mating components are disconnected from each other such that said other lead of said alarm module makes electrical connection with said other corresponding terminal of said battery

21. The apparatus with a mating assembly of claim 1, wherein said alarm module is an electromagnetic buzzer and said battery is a zinc-air battery.

22. The apparatus with a mating assembly of claim 1, wherein said force applicator is one of a compression spring, a tension spring or a permanent magnet.

23. The apparatus with a mating assembly of claim 1, wherein said alarm module is one of a piezoelectric buzzer, a electromagnetic buzzer, a LED module or an RF transmitter module.

24. The apparatus with a mating assembly of claim 1, wherein battery contacts are attached to said leads of said alarm module such that said battery contacts are used to make electrical contact with corresponding contacts of said battery.

25. The apparatus with a mating assembly of claim 1, wherein said mating assembly comprises one of a mating slip connector assembly or mating mechanical container parts.

26. The apparatus with a mating assembly of claim 6, wherein said slip connectors may be one of fluids, electrical or optical type.
